(19) United States
(12) Patent Application Publication
(10) Pub. No.: US 2008/0161555 A1
(43) Pub. Date: Jul. 3, 2008

(54) Purification of Tacrolimus on Supports of Vegetable Origin

(75) Inventors: Luca Morra, Avigliana (TO) (IT); Jacopo Roletto, Torino (IT); Giovanni Lazzari, Settimo Torinese (IT); Walter Cabri, Rozzano (IT); Paolo Paissoni, Druento (IT)

Correspondence Address: ROTHWELL, FIGG, ERNST & MANBECK, P.C., 1425 K STREET, N.W., SUITE 800, WASHINGTON, DC, 20005, US

(73) Assignee: ANTIBIOTICOS S.p.A., Rodano, MI (US)

(21) Appl. No.: 11/997,799

(22) PCT Filed: Jul. 10, 2006
(86) PCT No.: PCT/EP2006/006722

§ 371 Date: Mar. 21, 2008

(30) Foreign Application Priority Data
Aug. 5, 2005 (IT) MI2005A001549

(51) Int. Cl. C07D 267/00

(52) U.S. Cl. 540/455

(57) ABSTRACT

The invention relates to a process for the purification of tacrolimus comprising contacting crude tacrolimus with a silver salt dissolved in a water/organic solvent mixture and with a carrier of vegetable origin, separating the mixture from the carrier and recovering purified tacrolimus.

### FIELD OF THE INVENTION

The present invention relates in general to immunosuppressant and antimicrobial tricyclic macrolides, in particular to a process for the recovery and purification of tacrolimus (I)

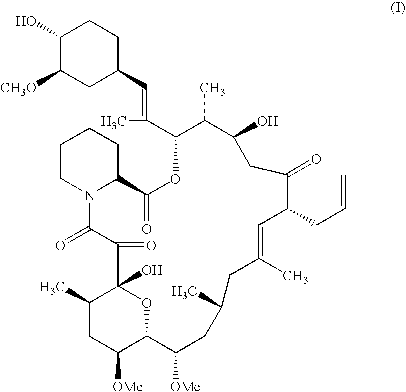

### TECHNOLOGICAL BACKGROUND

Tacrolimus (I) (17-allyl-1,14-dihydroxy-12-[2-(4-hydroxy-3-methoxycyclohexyl)-1-methylvinyl]-23,25-dimethoxy-13,19,21,27-tetramethyl-11,28-dioxy-4-azatricyclo-[22.3.1.0^{4,9}]octacos-18-ene-2,3,10,16-tetraone) is a tricyclic macrolide produced through fermentation of Streptomyces sp. Tacrolimus is used in the treatment of transplant rejection crisis, autoimmune diseases, infective diseases and the like.

EP 0184162 discloses a process for the preparation of tacrolimus and derivatives thereof through fermentation and chemical synthesis. In particular, the fermentation with Streptomyces sp. produces, besides tacrolimus, also the 17-ethyl derivative (II) (17-ethyl-1,14-dihydroxy-12-[2-(4-hydroxy-3-methoxycyclohexyl)-1-methylvinyl]-23,25-dimethoxy-13,19,21,27-tetramethyl-11,28-dioxy-4-azatricyclo-[22.3.1.0^{4,9}]octacos-18-ene-2,3,10,16-tetraone), commonly known as FK520

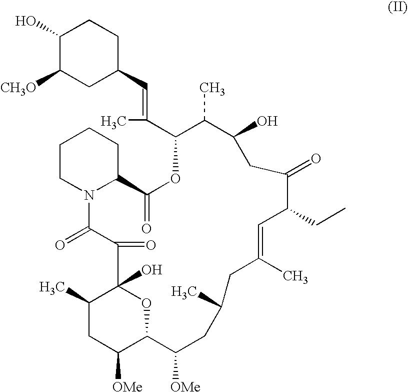

and the 17-propyl derivative (III) (17-propyl-1,14-dihydroxy-12-[2-(4-hydroxy-3-methoxycyclohexyl)-1-methylvinyl]-23,25-dimethoxy-13,19,21,27-tetramethyl-11,28-dioxy-4-azatricyclo-[22.3.1.0^{4,9}]octacos-18-ene-2,3,10,16-tetraone)

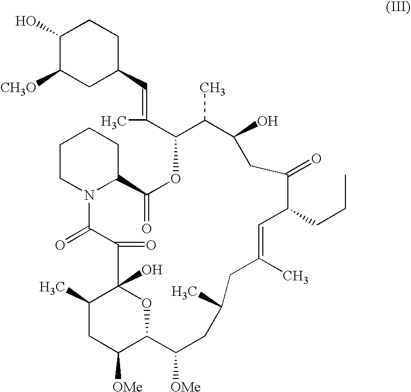

Further to the chemico-physical characterization of tacrolimus and related by-products, in EP 0184162 extraction, purification and recovery methods are also disclosed. In particular, recovery from fermentation broths is accomplished by means of known extraction techniques, such as: use of solvents suitable for extracting the activity from broths or mycelia; adsorption/elution with anionic and cationic exchange resins, or non-ionic absorbent resins; purification on conventional chromatographic supports, such as silica gel, allumina and cellulose; decolouration with active carbon, crystallization and recrystallization.

According to EP 0184162, the process for the extraction and recovery of tacrolimus and related by-products from fermentation broths comprises the following steps:
extraction of the mycelium and/or fermentation broth with solvents (for example acetone and methanol);
purification on non ionic absorbent resin (in particular HP-20);
evaporation of the purified solution to an oil;
purification on silica gel (in particular grade 12 silica gel, Fuji Devison Co.), repeated twice or thrice to obtain a product in the form of powder;
purification by preparative HPLC for the separation of the above described impurities.

The purification steps on non-ionic absorbent resin and those on silica gel are aimed at removing most compounds deriving from the fermentation broth (substances produced by the microorganism during fermentation, inorganic salts and substances deriving from the starting materials, whereas impurities (II) and (III) are separated by means of purification on preparative HPLC, which, however, has poor productivity and applicability on an industrial scale.

U.S. Pat. No. 6,492,513 discloses the purification of tacrolimus from impurities (II) and (III) by cationic-exchange resins pretreated with silver salts (in particular silver nitrate). The use of silver salts in the separation of cis-trans isomers of unsaturated aliphatic acids with the same number of carbon atoms is known in literature (J. Chromatography, 149 (1978) 417-430). The silver salts form π-complexes with unsaturated compounds that are thus separated depending on their conformation. With the process of U.S. Pat. No. 6,492,513 tacrolimus (which has an unsaturated 17-allyl side chain) is separated from the two impurities with a 17-saturated side chain, since tacrolimus is more tightly retained on the cationic-exchange resin than the two impurities, as it forms a silver complex.

Finally, U.S. Pat. No. 6,576,135 discloses the separation of tacrolimus from impurities (II) and (III) by means non-ionic absorbent resins.

### DETAILED DISCLOSURE OF THE INVENTION

It has now been found that tacrolimus can be conveniently purified from impurities (II) and (III) as π-complex with silver ions (IV)

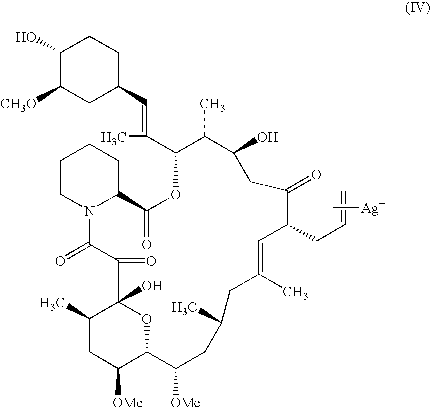

through the use of supports of vegetable origin.

Accordingly, the invention relates to a process for the purification of tacrolimus comprising contacting crude tacrolimus with a silver salt dissolved in a water-organic solvent mixture and with a carrier of vegetable origin selected from cellulose (such as ARBOCELL BC 200, J. Rettenmaier & Sohn or SOLKA FLOC, Dicalite), modified cellulose (such as Methocel, Dow Chemical), starches, modified starches, natural polymers having simple carbohydrates as monomers and carbon. In this way, the impurities are retained on the carrier of vegetable origin, whereas the complex tacrolimus-silver ions is eluted in the aqueous phase. After elution of the solvent mixture from the carrier, tacrolimus can be recovered treating the aqueous phase with an organic solvent in which tacrolimus is soluble.

Therefore, according to a first embodiment, the process of the invention comprises:
a. contacting crude tacrolimus with a silver salt dissolved in a water-organic solvent mixture and with a carrier of vegetable origin selected from cellulose, modified cellulose, starches, modified starches, natural polymers having simple carbohydrates as monomers and carbon;
b. separation of the water/organic solvent mixture from the carrier;
c. recovery of tacrolimus from the aqueous phase.

In particular, recovery of tacrolimus from the aqueous phase can accomplished by means of:
c1. removal of the organic solvent;
c2. extraction of the aqueous phase with an organic solvent in which tacrolimus is soluble;
c3. separation of the organic phase from the aqueous one and recovery of purified tacrolimus through evaporation of the solvent.

According to a preferred embodiment of the invention, tacrolimus purified in this way can be subjected again to the process, using the same carrier of vegetable origin as the first purification step, or a different one. For example, during the first purification step cellulose can be used, whereas during the second purification step carbon can be used, or vice-versa.

For the purposes of the present description, the expression “organic solvent” means a water-miscible or non-miscibile solvent selected from ketones, alcohols, aliphatic and alicyclic hydrocarbons; preferred solvents are acetone, methanol, n-hexane and cyclohexane.

An organic solvent in which tacrolimus is soluble and which is used for the recovery of tacrolimus from the aqueous phase (step c2) is a non water-miscible organic solvent, for example ethyl acetate, methyl-ethyl-ketone, ethyl ether, dichloromethane, preferably ethyl acetate. In more detail, step c is carried out as follows: the mixture containing the tacrolimus-silver π-complex is concentrated under vacuum to remove the organic solvent (step c1) and subsequently extracted with 0.5-3 volumes of organic solvent in which tacrolimus is soluble (step c2). The organic phase is washed with 1 volume of deionized water for 2-3 times and subsequently concentrated to a small volume (step c3).

The aqueous phase obtained at step c3, which contains silver salts, can be recovered and recycled for the preparation of the tacrolimus-silver ions complex.

The weight amount of the carrier of vegetable origin is 3÷100 times compared with the weight of tacrolimus, preferably 15÷25 times. Where the carrier of vegetable origin is carbon, it is used in an amount of 3÷50 times compared with the weight of tacrolimus, preferably 5÷15 times. In this case, the complex tacrolimus-silver ions in the water-organic solvent mixture is loaded onto a column containing carbon and equilibrated with a mixture consisting of water and the same organic solvent as the mixture containing the complex. The elution flow rate is 0.5÷10 vol/h depending on the volume of carbon used, preferably 3.0÷5 vol/h. The volume of the washing solution is 2÷10 times compared with the volume of carbon, preferably 3÷8 times. The eluate is then recovered and subjected to the above described steps a-d.

The silver ions which form the complex subjected to purification are released from silver salts, preferably silver nitrate or silver perchlorate. The concentration of silver ions preferably ranges from 0.05 to 1.30 mol/l, most preferably from 0.20 to 0.30 mol/l. The tacrolimus-ions silver complex to be subjected to the first purification cycle is prepared dissolving the fermentation product from Streptomyces sp in an organic solvent in which tacrolimus is soluble, preferably selected from ethyl acetate, methanol and acetonitrile. Carriers other than carbon are added to the solution, thereafter the organic solvent is evaporated off and the obtained solid is extracted with the water-organic solvent mixture containing a silver salt. The amount of organic solvent in the extraction mixture ranges from 0 to 60% compared with the volume of the aqueous solution, preferably from 0 to 20%. The weight amount of the extraction mixture is 50÷500 times compared with the weight of tacrolimus. The solid-liquid extraction can be repeated up to 5 times, so as to obtain a molar yield of extraction of purified tacrolimus higher than 90%.

On the other hand, when the carrier is carbon, the fermentation product is directly treated with a water-organic solvent mixture containing a silver salt, then loaded on the column.

According to a further embodiment, the process of the invention can also comprise a chromatographic purification on a non ionic resin, according to what described, for example, in EP 0184162. The resin is usually selected from commercially available absorbent resins, preferably from Mitsubishi Chemical Corporation (SP200 or SP800) or Rohm and Haas (series XAD). This additional step can be performed either before or after the purifications with supports of vegetable origin. According to a particularly preferred embodiment, this additional step is performed before, as described in the following in greater detail.

The fermentation broth or mycelium, suitably filtered, is extracted with organic solvents in which tacrolimus is soluble, preferably ketones or alcohols, more preferably acetone or methanol, and the extraction product is subjected to absorption chromatography on a non ionic absorbent resin, to purify tacrolimus and impurities (II) and (III) from other compounds contained in the fermentation broth, such as substances released by the microrganism during fermentation, inorganic salts and substances deriving from the starting materials.

The resulting product is dissolved in a water-organic solvent mixture containing a silver salt subjected to the process of the invention. Preferably, the mixture containing the tacrolimus-silver ions complex is first purified on carbon, as described above, then the purification process is repeated using another carrier of vegetable origin, preferably cellulose.

Pure tacrolimus obtained with the process of the invention is subjected to crystallization with known methods; usually the product is dissolved in an organic solvent, preferably acetonitrile, and precipitated as monohydrate crystal, by addition of deionized water. The resulting crystal is characterized by high purity (HPLC % area >99% according to the HPLC method reported in Y. Namiki et al. Chromatographia Vol. 40, No 5/6 Mar. 1995).

The process of the invention is particularly advantageous compared with known processes, both from the point of view of productivity and from that of the cost of the finished product.

As regards productivity, the process of the invention does not require repeated chromatographic purifications on normal phase silica gel. Said purifications, required by the extraction/purification procedures disclosed in literature, involve the use of remarkable amounts of solvents and silica gel and prolonged times.

The cost of the finished product is indeed lower than that of the product obtainable with known methods, as the process of the invention comprises the use of supports of vegetable origin available on the market at a cost much lower than the chromatographic supports used in the procedures disclosed in the literature. Moreover, according to the process of the invention, the aqueous solution containing silver ions can be fully recycled, which allows to keep environmental impact low and to limit the incidence of the cost of the silver salt on that of the finished product.

The invention will be now illustrated in more detail by means of some examples.

### EXAMPLES

### Example 1

### Extraction and Purification on Absorbent Resin

50 liters of fermentation broth are added with 50 liters of acetone and 1 kg of filtration adjuvant (Dicalite). After stirring at room temperature for one hour, the slurry is filtered. The resulting clarified solution is absorbed on 2 liters of absorbent resin XAD16 (Rohm and Haas). The activity is eluted with 6 liters of water/acetone 25/75. The resulting solution is concentrated to remove acetone. The aqueous phase (1.5 liters) is extracted with 1.5 liters of ethyl acetate. The phases are separated and the organic phase is concentrated to an oil.

### Example 2

### Purification on Granular Carbon

The oily phase is added with 180 ml of a 50/50 water/acetone solution containing 13.5 g of AgNO₃. The resulting solution is percolated onto a column containing 100 ml of granular carbon GAC 1240 PLUS (CECA ITALIANA), previously conditioned with 150 ml of a 50/50 water/acetone solution containing 11.25 g of AgNO₃. Thereafter, 400 ml of a 50/50 water/acetone solution containing 30 g of AgNO₃ are eluted through the column. The resulting solution is evaporated to a volume of 350 ml. 350 ml of ethyl acetate are added, the phases are separated and the aqueous phase is recycled, whereas the organic phase is processed according to example 3.

### Example 3

### Purification on Cellulose

The organic phase from example 2 is added with 160 g of cellulose SOLKA FLOC (DICALITE). The suspension is evaporated until complete removal of the organic solvent. The resulting solid is added with 2 liters of a 75/25 water/n-hexane solution containing 111.5 g of AgNO₃ and stirred for 30 minutes at room temperature, thereafter the solid is filtered. Extraction and filtration are repeated 4 times and the phases are separated. The aqueous phase is added with 1 l of ethyl acetate and the phases are separated. The aqueous phase is recycled, whereas the organic phase is washed thrice with 1 volume of deionized water, compared with the volume of the organic phase, and subsequently concentrated to obtain a white solid (9.2 g).

### Example 4

### Crystallization of Tacrolimus Monohydrate

The solid product (9.2 g, containing 8.5 g of tacrolimus) is dissolved in 700 ml of acetonitrile. 1200 ml of deionized water are slowly added (1-2 hours) at 25° C. and the solution is cooled to 5° C., allowed to stand at 5° C. for 12-14 hours and then filtered. 7.0 grams of highly pure tacrolimus (HPLC % area >99%) are obtained.

### Example 5

### Purification on Granular Carbon with Recycle of the Aqueous Solution of AgNO₃

Residual organic solvents are stripped off from the aqueous phase from example 2 to a 290 ml solution, which is then added with 290 ml of acetone.

The oily phase obtained according to the procedure described in example 1 is added with 180 ml of the recycled water/acetone solution. The resulting solution is loaded onto a column containing 100 ml of granular carbon GAC 1240 PLUS (from CECA ITALIANA) previously conditioned with 150 ml of a 50/50 water/acetone solution containing 11.25 g of AgNO₃. Thereafter, 400 ml of the recycled water/acetone solution are eluted. The resulting solution is evaporated to 350 ml and added with 350 ml of ethyl acetate, then the phases are separated and the aqueous one is recycled, whereas the organic one is worked up according to what reported in example 6.

### Example 6

### Purification on Cellulose with Recycle of the Aqueous AgNO₃ Solution

Residual organic solvents are stripped off the aqueous phase to be recycled obtained according to example 3, to give 6 l of a solution which is added with 2 l of n-hexane.

The organic phase from example 5 is added with 160 g of cellulose SOLKA FLOC (DICALITE). The suspension is evaporated until the organic solvent is removed. The resulting solid is added with 2 liters of the water/n-hexane solution obtained from the recycle and stirred for 30 minutes at room temperature, then the solid is filtered. Extraction and filtration are repeated 4 times and the phases are separated. The aqueous phase is added with 1 l of ethyl acetate and the phases are separated. The aqueous phase is recycled, whereas the organic phase is washed thrice with 1 volume of deionized water compared with the volume of the organic phase and subsequently concentrated to obtain a white solid product (9.2 g).

### Example 7

### Crystallization of Tacrolimus Monohydrate

The solid product (9.2 g, containing 8.5 g of tacrolimus) is dissolved in 700 ml of acetonitrile. 1200 ml of deionized water at 25° C. are slowly added (1-2 hours) and the solution is cooled to 5° C., then allowed to stand at 5° C. for 12-14 hours and is filtered. 7.0 grams of highly pure tacrolimus (HPLC % area >99%) are obtained.

1. A process for the purification of tacrolimus (I) 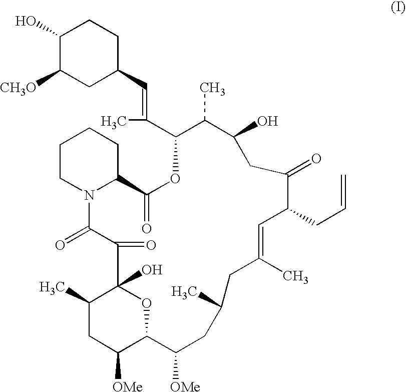
comprising
a. contacting crude tacrolimus with a silver salt dissolved in a water-organic solvent mixture and with a carrier of vegetable origin selected from cellulose, modified cellulose, starches, modified starches, natural polymers having simple carbohydrates as monomers and carbon;
b. separating the water/organic solvent mixture from the carrier;
c. recovering tacrolimus from the aqueous phase.

2. A process as claimed in claim 1 wherein the recovery of tacrolimus from the aqueous phase is accomplished by means of:
c1. removal of the organic solvent;
c2. extraction of the aqueous phase with an organic solvent in which tacrolimus is soluble;
c3. separation of the organic phase from the aqueous one and recovery of purified tacrolimus through evaporation of the solvent.

3. A process as claimed in claim 1 further comprising:
d. treating purified tacrolimus with a water/solvent mixture containing a silver salt and repeating steps a-c.

4. A process according to claim 1 in which the carrier is cellulose.

5. A process according to claim 1 in which the carrier is carbon.

6. A process as claimed in claim 3 in which step a) is carried out the first time with carbon and the second time with cellulose.

7. A process as claimed in claim 3 in which step a) is carried out the first time with cellulose and the second time with carbon.

8. A process according to claim 1 in which the solvent is an alcohol or a ketone.

9. A process as claimed in claim 8 in which the solvent is methanol or acetone.

10. A process according to claim 1 in which the organic solvent is an aliphatic or alicyclic hydrocarbon.

11. A process as claimed in claim 10 in which the solvent is n-hexane or cyclohexane.

12. A process as claimed in claim 2 in which the organic solvent in which tacrolimus is soluble is ethyl acetate.

13. A process as claimed in claim 2 in which the aqueous solution obtained from the extraction of step d) is recycled to step a).

14. A process according to claim 1 further comprising chromatographic purification of tacrolimus on a non ionic resin.
